(19) United States
(12) Patent Application Publication
(10) Pub. No.: US 2020/0110145 A1
(43) Pub. Date: Apr. 9, 2020

(54) DETECTION OF A WRONGLY DETECTED MOTION

(71) Applicant: Siemens Healthcare GmbH, Erlangen (DE)

(72) Inventor: Mario Zeller, Erlangen (DE)

(21) Appl. No.: 16/592,751

(22) Filed: Oct. 3, 2019

(30) Foreign Application Priority Data
Oct. 4, 2018 (EP) 18198623.3

(51) Int. Cl. G01R 33/565; A61B 5/055; A61B 5/113; G06T 7/20; A61B 5/11; A61B 5/00

(52) U.S. Cl. CPC G01R 33/56509; A61B 5/055; A61B 5/7207; G06T 7/20; A61B 5/1126; A61B 5/113

(57) ABSTRACT

A method for correcting a wrongly detected motion of an object under examination during an MR imaging procedure at which MR signals from a first part of the object are detected in order to generate MR images of the first part. A first motion signal of the object is detected during MR signal detection, and a second motion signal of the objet is detected during MR signal detection. The first motion signal is correlated with the second motion signal in order to separate an actual motion of the first part from the wrongly detected motion. A presence of the actual motion and the wrongly detected motion in the two motion signals is different. A motion compensation is carried out on the generated MR images in which substantially only the actual motion and not the wrongly detected motion of the object under examination is corrected in the detected MR signals.

### PRIORITY

This application claims the benefit of EP 18198623.3, filed on Oct. 4, 2018, which is hereby incorporated by reference in its entirety.

### TECHNICAL FIELD

The present embodiments relate to detecting a wrongly detected motion of an object under examination during a magnetic resonance (MR) imaging procedure.

### BACKGROUND

Magnetic resonance imaging (MRI) is an imaging modality allowing a high-resolution generation of images of an object under examination, such as a human being. Movements during a magnetic resonance (MR) image acquisition (e.g., respiratory movements of the object under examination) may result in artifacts (e.g., types known as ghosting, blurring, and/or loss of intensity in the generated images). Numerous techniques are known for reducing artifacts as a result of respiratory motion (e.g., as disclosed in US 20160245888 A1, US 20170160367 A1, or US 20170160364 A1).

The breathing of the person under examination may also be detected using external sensors such as a pneumatic cuff or based on signal processing of image signals generated by a camera monitoring the examined person. A camera based motion correction identifies the motion in the generated images of the camera and tries to correct the motion in the image. These camera based motion correction techniques may rely on a detection of a marker with a Moiré pattern mounted on top of a nose of the examined person whose head motion is supposed to be corrected. The pattern is affected by a wrongly detected motion. This wrongly detected motion has the effect that the image quality of the generated motion compensated MR images that are generated based on the acquired MR signals is often inferior to acquisitions without any motion correction.

The root cause of this effect seems to be twofold: a) The examined person feels irritated by the marker sticking on the nose and thus slightly moves the nose (e.g., the alas of the nose while the head is not moving and remains still); b) the alas of the nose (e.g., the nose itself is slightly moving during the inhaling or exhaling of the patient during respiration).

Since the camera detects the motion of the nose marker and assumes that the motion is rigid, the acquired raw data is wrongly corrected for the whole head. However, this problem does not only occur in the case where a marker is attached to the user's nose or head. Also, in other motion correction techniques without the marker, the head of the examined person is monitored, and by post processing the detected images of the face of the person, the movement of the nose or any other part during inhaling or exhaling may be detected while the head itself that is examined is not moving at all.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a wrongly detected motion of an object under examination is differentiated from an actual real motion of the object under examination during an acquisition of magnetic resonance (MR) signals.

According to a first aspect, a method for detecting a wrongly detected motion of an object under examination during an MR imaging procedure, at which the MR signals are detected from a first part of the object under examination in order to generate MR images of the first part, is provided. A first motion signal of the object under examination is detected during the MR signal detection. The first motion signal is independent of a position from which the MR signals are originating in the object under examination. A second motion signal of the object under examination is detected during MR signal detection, and the second motion signal is independent of a position from which the MR signals are originating in the object under examination. The first motion signal is then correlated with the second motion signal in order to separate an actual motion of the first part of the object under examination during the MR signal detection from the wrongly detected motion in which the first part of the object under examination is not moving, but a second part different from the first part of the object under examination is moving. The presence of the actual motion and the wrongly detected motion in the two motion signals is different. Further, a motion compensation is carried out on the generated MR signals in which substantially only the actual motion and not the wrongly detected motion of the object under examination is corrected in the detected MR signals.

With the detection of the two motion signals, which do not depend from the part of the object under examination the MR signals come from, it is possible to differentiate between an actual or real motion and the wrongly detected motion that occurs in a part of the object under examination but not in the part of the object from which a major part of the MR images are generated. As the first motion signal and the second motion signal are such that the wrongly detected motion may be present in both of the two signals, whereas the actual motion may be present in only one of the two signals, it is possible to differentiate between these two different motions. In another embodiment, one of the two motion signals carries or includes the wrongly detected motion and the actual motion (thus both motion signals), whereas the other of the two motion signals carries either the wrongly detected or the actual motion. Thus, the actual motion may be separated from the wrongly detected motion. It is possible to remove the signal influence from the respiratory motion in the detected motion signals during an MR head scan, so that only the remaining motion, the real or actual motion, is detected, which may be used to carry out a motion compensation in the generated MR images.

In one embodiment, the detected first motion signal and the detected second motion signal are independent of the acquisition of the MR signals used to generate the MR images. The detected first motion signal and the second motion signal may be detected during the entire examination of the object under examination, the patient. The signal detection of the motion signals works irrespectively of the fact whether an imaging sequence that is supposed to be corrected is currently running or whether adjustment data, localizer data, or imaging data for a preceding measurement is acquired, or the person examined is currently only instructed and no data is acquired at all. All these periods where no MR signals are detected for the MR images to be corrected may be used for learning and continuously updating a correlation pattern generated between the second motion signal and the first motion signal. When the first motion signal and the second motion signals are detected and if there is no analytical or empiric relation between the two motion signals, which is constant for the different image acquisitions, a learning phase may be introduced, in which the relation between the two signals is learnt and which allows the wrongly detected motion to be separated from the actual or real motion. This learning may happen continuously before the image acquisition for the image to be corrected is started.

The first motion signal and the second motion signal may be signals detected from two different sensors configured to detect different signals of the object under examination. By way of example, detecting one of the first motion signal or the second motion signal may include the acquisition of picture data of the object under examination with a camera and post processing the acquired picture data. The detecting of the first motion signal or the second motion signal may also include acquiring sensor data from a sensor configured to detect the respiratory motion of the object under examination such as a respiratory sensor (e.g., a breathing belt, etc.).

Further, for detecting the first motion signal or the second motion signal, it is possible to detect a pilot tone signal transmitted by a pilot tone transmitter. This pilot tone signal is detected in different coil channels of at least one receiving coil used to detect the MR signals. This pilot tone signal is within a first frequency range that is detectable by the at least one receiving coil, but the pilot tone signal is outside a second frequency range in which the MR signals that are used to generate the MR images are present and detected. By post processing the pilot tone signal of the different coil channels, the wrongly detected motion and the actual motion may be identified.

By way of example, the first motion signal contains the breathing curve as a surrogate that correlates with the wrongly detected motion (e.g., occurring at the nose). The second signal originating from the coils signal channels from the pilot tone includes in one channel the wrongly detected motion (e.g., of the nose), whereas the other channels only contain the true motion

When the pilot tone signal is used, correlating the first motion signal with the second motion signal may provide that it is detected whether changes in the pilot tone signal are detected in the different channels of the at least one receiving coil. The actual motion may then be detected in the first motion signal or the second motion signal when the changes in the pilot tone signal are detected in all channels of the at least one receiving coil. When the changes are only detected on one or several but not all channels, it may be deduced that the signal changes in the channels where the changes are present are due to the wrongly detected motion.

In this context, it the actual motion may be detected in the pilot tone signal as the first motion signal when the same changes of the pilot tone signal are detected in all sections of the at least one receiving coil. The wrongly detected motion is then present in the other motion signal, the second motion signal, but not in the first motion signal. The pilot tone may be a signal that may be generated by the MR system, or may be generated by a transmitter added to the receiving coils or any other appropriate location near the MR system. The pilot tone has a frequency that is different from the signals and outside the frequency range used to excite the spins of the object under examination. Nevertheless, the pilot tone signal is modulated by the object under examination and may be detected by the receiving coils, and if all the sections of the receiving coil or the receiving coils detect the same changes in the pilot tone signal, it may be deduced that the object under examination in total has moved. If only a single part of the object under examination, such as the nose or the mouth, is moving, changes in the pilot tone signal are not detected in all channels of the receiving coils.

The object under examination may be a head, and the motion compensation may include the compensation of the rigid head movements occurring during the MR signal detection. The head movement may, for example, include six degrees of freedom, three translational coordinates, and three rotational coordinates.

The wrongly detected motion may be due to a respiration induced movement of a part of the nose present in both the first motion signal and the second motion signal. The actual motion signal is only present in one signal of the first motion signal and the second motion signal. The respiration induced movement may thus be detected by correlating the two motion signals and may be removed in order to carry out the motion compensation only based on the actual rigid movement of the head.

Further, a corresponding motion correction module configured to correct the wrongly detected motion of the object under examination is provided. The motion correction module may include a memory and at least one processing unit (e.g., at least one processor). The memory includes instructions executable by the at least one processing unit. The motion correction module its operative to function as discussed above or as discussed in further detail below.

According to a further aspect, a computer program including program code to be executed by at least one processing unit of the motion correction module is provided. Execution of the program code causes the at least one processing unit to execute a method as discussed above or as discussed in further detail below.

Additionally, a non-transitory computer-readable data storage medium encoded with programming instructions is provided. The data storage medium is loaded into a motion correction module, as discussed above. The execution of the programming instructions cause the at least one motion correction module to execute a method, as discussed above, or as discussed in further detail below.

The features mentioned above and features yet to be explained below may be used not only in the respective combinations indicated, but also in other combinations without departing from the scope of the present invention. Features of the above mentioned aspects and embodiments described below may be combined with each other in other combinations unless explicitly mentioned otherwise.

### BRIEF DESCRIPTION OF THE DRAWINGS

The foregoing and additional features and effects of the application will become apparent from the following detailed description, when read in conjunction with the accompanying drawings, in which like reference numerals refer to like elements.

FIG. 1 shows a schematic view of one embodiment of a magnetic resonance (MR) system.

FIG. 2 shows a more detailed schematic view of a part of the system of FIG. 1.

FIG. 3 shows a schematic view of a flowchart of one embodiment of a method carried out by a motion correction module used in the MR system of FIG. 1.

FIG. 4 shows an example schematic architecture view of a motion correction module configured to detect a wrongly detected motion of an object during MR signal acquisition.

### DETAILED DESCRIPTION

In the following, embodiments will be described in detail with reference to the accompanying drawings. The following description of embodiments is not to be taken in a limiting sense. The scope of the invention is not intended to be limited by the embodiments described hereinafter or by the drawings, which are to be illustrative only.

The drawings are to be regarded as being schematic representations, and elements illustrated in the drawings are not necessarily shown to scale. Rather, the various elements are represented such that function and general purpose becomes apparent to a person skilled in the art. Any connection or coupling between functional blocks, devices, and/or components of physical or functional units shown in the drawings and described hereinafter may be implemented by an indirect connection or coupling. A coupling between components may be established over a wired or wireless connection. Functional blocks may be implemented in hardware, software, firmware, or any combination thereof.

In the following, a procedure is explained in more detail how a wrongly detected motion may be detected in an examination of an object where magnetic resonance (MR) signals are detected from the object under examination. The idea described below especially allows a differentiation between an actual motion that influences the MR signal detection and a motion of a part of the examined object, which substantially does not influence the detected MR signals or the generated MR images.

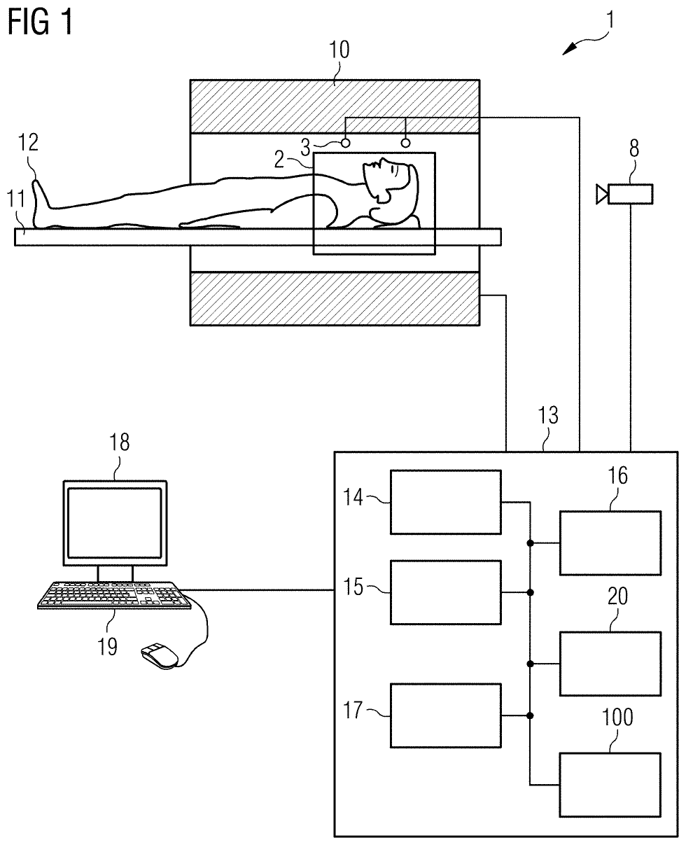

FIG. 1 shows a schematic view of one embodiment of an MR system 1 that includes a magnet 10 generating a polarization field BO. An object under examination 12 lying on a table 11 is moved into a center of the MR system 1 where MR signals after RF excitation may be detected by a receiving coil 2. The receiving coil may include different coil sections. Each of the different coil sections is associated with a corresponding detection channel. By applying RF pulses and magnetic field gradients, the nuclear spins in the object 12 (e.g., in a part located in the receiving coil 2) are exited, and the currents induced by the mechanization are detected. How MR images are generated and how the MR signals are detected using a sequence of RF pulses and the sequence of magnetic field gradients are known in the art, so that a detailed explanation thereof is omitted.

The MR system includes a control module 13 that is used for controlling the MR system. The control module 13 includes a gradient control unit 14 for controlling and switching the magnetic field gradients, and an RF control unit 15 for controlling and generating the RF pulses for the imaging sequences. An image sequence control unit 16 that controls the sequence of the applied RF pulses and magnetic field gradients and thus controls the gradient control unit 14 and the RF control unit 15 is provided. In a memory 17, computer programs needed for operating the MR system and the imaging sequences necessary for generating the MR images may be stored together with the generated MR images. The generated MR images may be displayed on a display 18. Input unit 19 used by a user of the MR system to control the functioning of the MR system is also provided. A processing unit 20 (e.g., a processor) may coordinate the operation of the different functional units shown in FIG. 1 and may include one or more processors that may carry out instructions stored on the memory 17. The memory includes the program code to be executed by the processing unit 20 or by a motion correction module 100 that is configured to correct the motion induced artifacts in the generated MR image. The motion correction module 100 is configured, for example, to differentiate between an actual motion of the object under examination, the person 12, and the wrongly detected motion that is a motion of the part of the examined object that is, however, not the part that is mainly displayed in the generated MR images and from which the MR signals are detected. In FIG. 1, a camera 8 that is configured to acquire picture data from the object under examination is shown. The generated picture data may be acquired with a frequency such that a movement of the part of the object under examination may be detected in the generated picture frames. By way of example, the frame rate of the generated picture frames may be between 1 and 50 frames per second.

The picture data may be post processed either by the processing unit 20 or by the motion correction module 100 in order to detect a motion of the person 12 during a time period when the MR signals are detected. Based on the acquired picture data, a first motion signal that describes the motion of the person 12 during image acquisition may be generated.

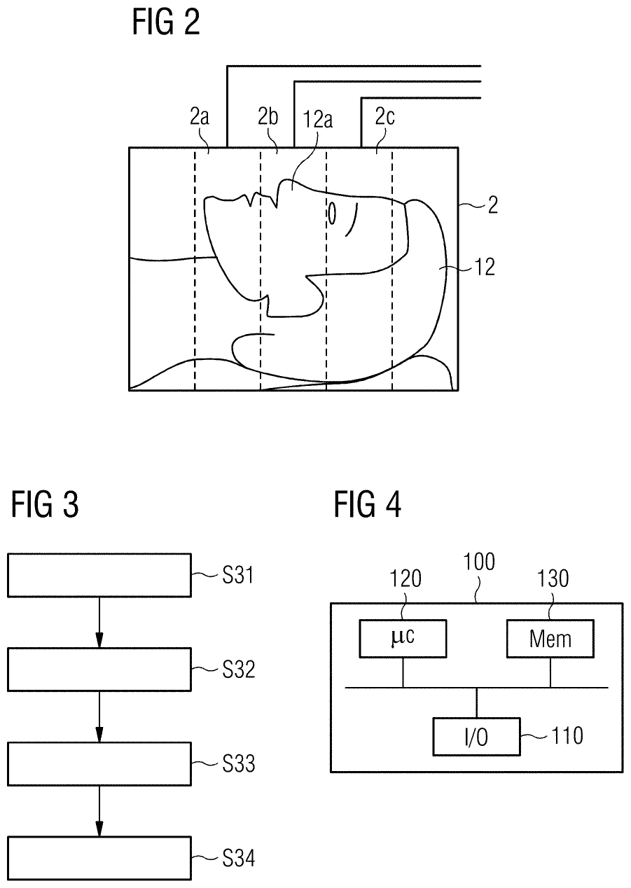

The RF control unit 15 may be further configured to act as a unit configured to generate a pilot tone that is transmitted by the MR system, either by a body coil (not shown) or by the indicated coil 2. This pilot tone signal is outside of the frequency range used for detecting the MR signal, but still within a frequency range that may be detected by the receiving coil, too. Further details about the generation and detection of pilot tone signals are disclosed in DE102015203385 A1. This pilot tone and the changes in the pilot tone occurring when the object under examination is moving may be used to generate a second motion signal that is deduced from the pilot tone signal that is detected in different coil channels of the receiving coil 2, as will be explained in more detail in connection with FIG. 2.

A further option to determine a motion signal is based on a respiratory sensor such as a pneumatic cuff. This respiratory sensor is not shown in the figure for the sake of clarity.

Accordingly, the system uses at least two different sensors provided in the MR system 10 to distinguish between the actual motion and the wrongly detected motion of the object under examination. These two motion signals are independent of the detected MR signal and are independent of the position of the detected MR signal. The two sensors are sensors that are not primarily used to generate the MR images. Three different options for the motion sensors were discussed above; however, other sensors such as radar sensors may be used to determine a movement or motion of any part of the object under examination. At least two of the motion signals generated by these sensors will be used to differentiate between the actual motion and the wrongly detected motion.

In connection with FIG. 2, the receiving coil 2 with different coil channels 2a, 2b and 2c is shown. In each coil channel, a separate MR image signal is detected, and the respiration of the patient 12 may lead to a movement of the alas 12a of the nose, shown in FIG. 2. A marker (not shown) may be mounted on top of the nose in order to improve the detection of the motion; however, the marker is not necessarily provided as the motion may also be detected from the acquired picture data alone by a post processing the images and by detecting a movement of the patient.

In the following, a number of options of how an actual movement of the head may be differentiated from a movement occurring only in a part of the nose that will be detected by the motion sensors discussed above, but will have substantially no influence on the generated MR images of the head (e.g., of the interior part of the head) are disclosed.

In one embodiment, the breathing signal detected by a respiratory sensor (e.g., the pilot tone sender or the breathing belt) may be used to determine and mask out the periodically detected motion trajectory caused by the movement of the part of the face (e.g., the nose) that is substantially not present in the generated MR images. In this context, it is possible to obtain the respiratory signal curve (e.g., first motion signal) and to obtain the x, y, z, and the corresponding rotation angle is detected using the camera 8 (e.g., second motion signal). Additionally, each coordinate may optionally be correlated with the respiratory signal curve during a learning phase, and a similarity pattern may be stored. Over several breathing cycles, the respiratory signal is correlated with each coordinate. When the correlation coefficient is similar over several time periods of the breathing cycle, the correlation coefficient may be used and stored as a reference coefficient for the corresponding coordinate. Later, during MR signal acquisition, the continuously determined correlation coefficients are determined. If the continuously determined correlation coefficients differ largely from the reference coefficient, it may be deduced that there is no respiratory influence on the motion signal, and no motion correction is carried out. The motion signal averaged over several breathing cycles may be used to generate a model difference signal having low noise components that may be subtracted from the wrongly detected motion signal. If needed, the model difference signal may be scaled by a dynamically determined scaling coefficient. Coordinates for which the correlation is very low may be excluded from the processing. Thus, each coordinate detected by the camera may be correlated with the respiratory signal curve during the imaging sequence, and the correlation coefficients may be compared with previously stored correlation coefficients. If the correlation is high and/or comparable to a similarity pattern calculated in an optional learning phase, the signal portion may be removed due to the respiratory motion from the camera coordinates, and only the remaining portion may be corrected. As mentioned above, the wrongly detected motion may be present in both motion signals, and the actual motion may be present in only one of the two signals.

In general, any kind of correlation between the two motion signals may be used. The correlation may be any kind of correlation and may include a subtraction of one of the motion signals from the others, a quotient of the two signals, etc.

As an alternative, instead of using a respiratory sensor, the pilot tone that is detected in all the different coil elements 2a, 2b, and 2c shown in FIG. 2 may be used. The pilot tone, as detected by the different coil segments, is used to determine whether a motion trajectory detected by the camera is detected in all coil segments 2a, 2b or 2c, or only in some of the coil segments close to the nose. By way of example, if the motion is detected in all coil segments, it may be deduced that a rigid motion of the head is occurring, which should be corrected. If, however, the main signal influence is occurring only in one or some of the coil segments, such as coil segment 2b shown in FIG. 2, it may be deduced that the motion is mainly due to the motion of a part of the nose. This wrongly detected motion may not be used to correct the motion of the complete head, as this would lead to a motion corrected image with a lower image quality compared to the non-motion compensated image.

In this implementation, the pilot tone signal may be detected from each coil segment, 2a, 2a, and 2c. Further, the translation and rotation coordinates are deduced from the camera data. Further, the x, y, z and the respective rotation angles may be directly determined from the coil signals, or each of the coil elements may be correlated directly with the camera coordinates. The coordinates may be correlated directly. In case of a high correlation, the camera signal is trusted and fully corrected. In case of a low correlation, the correction is skipped until a high correlation is obtained. As an alternative, the motion signal may be correlated from the camera with the signal from the respective coil segments. In case of a similar correlation in all channels, the correction is carried out, as this provides that a rigid head movement occurs. In the case that the correlation is high only in the channel segments close to the nose or the mouth, the correction may be skipped until a uniform correlation is achieved again.

Another source for the wrongly detected motion is a case where the person to be examined moves the hand to the part of the body to be examined (e.g., for scratching), but the part of the patient to be imaged does not really move.

An advantage of the use of the different motion signals as discussed above is that the different motion signals all receive data during the entire examination of the patient and work irrespective of whether the imaging sequence that is supposed to be correct is currently running, whether adjustment data, localizer data, or imaging data of another measurement are acquired, or if no data is acquired at all.

A detection and modification of the motion signal may be performed, for example, by a thresholding, machine learning, or a deep learning approach. Data of a previously acquired patient and feedback on the results by the MR system may be utilized to further improve the results. In a deep learning approach, correction patterns for the motion correction may be deduced from existing data sets. Further, there are methods for evaluating an image quality. Motion signal graphs, for which the motion correction worked well, may be used as appropriate training data for the learning approach. Curves with which a poor motion correction quality was obtained are used as “poor” training data. As an alternative to the automatic evaluation of images, it is possible that a user, after a measurement, marks the data set as good or appropriate training data or as poor training data. Further, if the MR image detection is repeated, it may be deduced that the former data set was a poor data set.

FIG. 3 summarizes some of the main acts discussed above carried out by the motion correction module 100 shown in FIG. 1. In act S31, a first motion signal of the object 12 is detected when the MR signal of the object 12 is detected. As discussed above, this first motion signal is independent of the position at which the MR signals are detected. The first motion signal may be a signal deduced from acquired picture data, may be a signal from a respiratory sensor such as a breathing belt, or may be a signal deduced from the detected pilot tone signal. In act S32, the second motion signal, which is also independent of the acquired MR images, is detected. In connection with act S31, three different options are discussed for the motion signal. If one of these signals is used in act S31, another signal of the remaining options is used in act S32 to detect the second motion signal that is also independent of the acquisition of the MR signals. In act S33, the first motion signal and the second motion signal are correlated in order to differentiate a motion of the part of the object 12 that is actually shown in the generated MR images and a part of the object that substantially has no influence on the generated MR images. As the presence of the actual and the wrongly detected motion signal is different in both motion signals, a separation is possible. The actual motion is separated from the wrongly detected motion. It is thus possible to carry out a motion compensation, act S34, in which only the actual motion is compensated for.

FIG. 4 shows a schematic architectural view of one embodiment of the motion correction module 100 shown in FIG. 1. The motion correction module 100 may be a separate entity provided in the control module 13 or may be implemented as part of the processing unit 20 shown in FIG. 1. If implemented as a separate entity, the motion correction module 100 includes an interface or input/output 110 that is used to receive the first motion signal, a second motion signal, and the picture data from the camera, any other data from the MR system, or data from outside the MR system. The interface 110 is used to transmit data to the other entities, such as the information about the differentiation between the actual and the wrongly detected motion. The entity also includes a processing unit 120 that is responsible for the operation of the motion correction module, as discussed above. The processing unit may include one or more processors used to carry out instructions stored on a memory 130. The memory 130 may be part of the memory 17 discussed above or may be a separate memory. The memory may include a read-only memory, a random access memory, mass storage, a hard disk, or the like. The memory may also include a suitable program code to be executed by the processing unit 120, so as to implement the above described functionalities in which the motion correction module is involved.

In summary, different sensors that are incorporated into MR systems are utilized. The data of the two motion sensors are correlated to improve the results of the sensor-based correction of movement. In general, the image quality of the motion compensated image is improved so that more confidence is provided into the results.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for detecting a wrongly detected motion of an object under examination during a magnetic resonance (MR) imaging procedure at which MR signals from a first part of the object under examination are detected in order to generate MR images of the first part, the method comprising:
detecting a first motion signal of the object under examination during MR signal detection, wherein the first motion signal is independent of a position from which the MR signals are originating in the object under examination;
detecting a second motion signal of the objet under examination during MR signal detection, wherein the second motion signal is independent of a position from which the MR signals are originating in the object under examination;
correlating the first motion signal with the second motion signal, such that an actual motion of the first part of the object under examination is separated during MR signal detection from the wrongly detected motion in which the first part of the object under examination is not moving but a second part different from the first part of the object under examination is moving, wherein a presence of the actual motion and the wrongly detected motion is different in the first motion signal and the second motion signal; and
carrying out a motion compensation on the generated MR images in which substantially only the actual motion and not the wrongly detected motion of the object under examination is corrected in the detected MR signals.

2. The method of claim 1, wherein the detected first motion signal and the detected second motion signal are independent of an acquisition of the MR signals used to generate the MR images.

3. The method of claim 1, wherein the first motion signal and the second motion signal are signals detected from two different sensors configured to detect different signals of the object under examination.

4. The method of claim 1, wherein detecting the first motion signal or detecting the second motion signal comprises acquiring picture data of the object under examination with a camera, and post-processing the acquired picture data.

5. The method of claim 1, wherein detecting the first motion signal or detecting the second motion signal comprises acquiring sensor data from a sensor configured to detect a respiratory motion of the object under examination.

6. The method of claim 1, wherein detecting the first motion signal or detecting the second motion signal comprises:
detecting a pilot tone signal transmitted by a pilot tone transmitter, wherein the pilot tone signal is detected in different coil channels of at least one receiving coil used to detect the MR signals and is within a first frequency range that is detectable by the at least one receiving coil but outside a second frequency range in which the MR signals of the object under examination are present; and
post-processing the pilot tone signal of the different coil channels.

7. The method of claim 6, wherein correlating the first motion signal with the second motion signal comprises detecting whether changes in the pilot tone signal are detected in the different channels of the at least one receiving coil, and
wherein the actual motion is detected in either the first motion signal or the second motion signal when the changes in the pilot tone signal are detected in all sections of the at least one receiving coil.

8. The method of claim 6, wherein the actual motion is detected in the pilot tone signal as the first motion signal when the same changes of the pilot tone signal are detected in all channels of the at least one receiving coil, and
wherein the wrongly detected motion is present in the second motion signal but not in the first motion signal.

9. The method of claim 7, wherein the wrongly detected motion is determined based on the fact that the changes in the pilot tone signal are present in at least one but not in all channels of the at least one receiving coil.

10. The method of claim 1, wherein the first motion signal and the second motion signal are detected, such that one motion signal of the first motion signal and the second motion signal comprises the actual motion and the wrongly detected motion, and the other motion signal of the first motion signal and the second motion signal comprises the actual motion or the wrongly detected motion.

11. The method of claim 1, wherein the object under examination is a head, and carrying out the motion compensation comprises compensating rigid head movements occurring during the MR signal detection.

12. The method of claim 11, wherein the wrongly detected motion is due to a respiration induced movement of a part of a nose present in both the first motion signal and the second motion signal,
wherein the actual motion is only present in one motion signal of the first motion signal and the second motion signal, and
wherein the respiration induced movement is removed, such that the motion compensation is carried out based on only the actual rigid movement of the head.

13. A motion correction module configured to correct a wrongly detected motion of an object under examination during a magnetic resonance (MR) imaging procedure at which MR signals from a first part of the object under examination are detected to generate MR images of the first part, the motion correction module comprising:
a memory and at least one processor, the memory storing instructions executable by the at least one processing unit to:
detect a first motion signal of the objet under examination during MR signal detection, wherein the first motion signal is independent of a position from which the MR signals are originating in the object under examination;
detect a second motion signal of the objet under examination during MR signal detection, wherein the second motion signal is independent of a position from which the MR signals are originating in the object under examination;
correlate the first motion signal with the second motion signal, such that an actual motion of the first part of the object under examination during MR signal detection is separated from the wrongly detected motion in which the first part of the object under examination is not moving but a second part different from the first part of the object under examination is moving, wherein a presence of the actual motion and the wrongly detected motion is different in the first motion signal and the second motion signal;
carry out a motion compensation on the generated MR images in which substantially only the actual motion and not the wrongly detected motion of the object under examination is corrected in the detected MR signals.

14. The motion correction module of claim 13, wherein the detected first motion signal and the detected second motion signal are independent of an acquisition of the MR signals used to generate the MR images.

15. In a non-transitory computer-readable storage medium that stores instructions executable by one or more processors to detect a wrongly detected motion of an object under examination during a magnetic resonance (MR) imaging procedure at which MR signals from a first part of the object under examination are detected in order to generate MR images of the first part, the instructions comprising:
detecting a first motion signal of the object under examination during MR signal detection, wherein the first motion signal is independent of a position from which the MR signals are originating in the object under examination;
detecting a second motion signal of the objet under examination during MR signal detection, wherein the second motion signal is independent of a position from which the MR signals are originating in the object under examination;
correlating the first motion signal with the second motion signal, such that an actual motion of the first part of the object under examination is separated during MR signal detection from the wrongly detected motion in which the first part of the object under examination is not moving but a second part different from the first part of the object under examination is moving, wherein a presence of the actual motion and the wrongly detected motion is different in the first motion signal and the second motion signal; and
carrying out a motion compensation on the generated MR images in which substantially only the actual motion and not the wrongly detected motion of the object under examination is corrected in the detected MR signals.
